SEQUENCE LISTING (6 sequences)

SEQ ID NO: 1 - protein, 12 aa, synthetic construct
HPDMFTRTHSHN

SEQ ID NO: 2 - protein, 12 aa, synthetic construct
HGLHSMHNKLQD

SEQ ID NO: 3 - protein, 12 aa, synthetic construct
GKPAVHYIHLRH

SEQ ID NO: 4 - protein, 12 aa, synthetic construct
HPFLHWNYGQRT

SEQ ID NO: 5 - protein, 12 aa, synthetic construct
GRKKRRQRRRPQ

SEQ ID NO: 6 - protein, 5 aa, synthetic construct
GGGSK

(19) United States
(12) Patent Application Publication
(10) Pub. No.: US 2026/0199529 A1
(43) Pub. Date: Jul. 16, 2026

(54) EPITHELIAL CELL ADHESION MOLECULE-SPECIFIC PEPTIDE CONJUGATES AND METHODS

(71) Applicant: REGENTS OF THE UNIVERSITY OF MICHIGAN, Ann Arbor, MI (US)

(72) Inventors: Thomas D. Wang, Ann Arbor, MI (US); Xiaoli Wu, Ann Arbor, MI (US)

(21) Appl. No.: 19/136,653

(22) PCT Filed: Dec. 12, 2023
(86) PCT No.: PCT/US23/83599

§ 371 Date: Jun. 6, 2025

Related U.S. Application Data
(60) Provisional application No. 63/387,214, filed on Dec. 13, 2022.

(51) Int. Cl. A61K 49/00; C07K 7/08

(52) U.S. Cl. CPC A61K 49/0056; A61K 49/0002; A61K 49/0032; C07K 7/08

(57) ABSTRACT

The disclosure relates to peptide conjugates specific for Epithelial cell adhesion molecule (EpCAM) and the use thereof to detect and treat epithelial cell-derived cancers such as intrahepatic cholangiocarcinoma (ICC), hepatocellular carcinoma (HCC), breast cancer, colon cancer and basal cell carcinoma of the skin. The disclosure also relates to methods to monitor the therapeutic response of treated patients by detecting expression of EpCAM.

### CROSS-REFERENCE TO RELATED APPLICATIONS

This application claims priority to Provisional Application No. 63/387,214, filed Dec. 13, 2022, which is incorporated herein by reference in its entirety.

### GOVERNMENT SUPPORT

This invention was made with government support under CA230669 awarded by the National Institutes of Health. The government has certain rights in the invention.

### FIELD

The disclosure relates to peptide conjugates specific for Epithelial cell adhesion molecule (EpCAM) and the use thereof to detect and treat epithelial cell-derived cancers such as intrahepatic cholangiocarcinoma (ICC), hepatocellular carcinoma (HCC), breast cancer, colon cancer and basal cell carcinoma of the skin. The disclosure also relates to methods to monitor the therapeutic response of treated patients by detecting expression of EpCAM.

### INCORPORATION BY REFERENCE OF THE SEQUENCE LISTING

This application contains, as a separate part of disclosure, a Sequence Listing in computer-readable form (Filename: 58623A_SeqListing.XML; 5,818 bytes; Created: Dec. 12, 2023) which is incorporated by reference herein in its entirety.

### BACKGROUND

Intrahepatic cholangiocarcinoma (ICC) is the second most common primary liver cancer, and accounts for up to 20% of all hepatic tumors.¹⁻³ This malignancy arises from the intrahepatic bile ducts, is aggressive in nature, has high mortality, and is associated with an overall 5-year survival rate<20%,⁴⁻⁶ Worldwide, the incidence of ICC is expected to increase by greater than ten-fold over the next 2 decades.⁷ Complete surgical resection remains the only viable therapy. Liver transplantation is recommended for only a subset of patients with early stage ICC following neoadjuvant chemoradiation therapy.⁸ This disease occurs most often in Asia, but is also increasing in western countries. Viral hepatitis (HBV and HCV) and primary sclerosing cholangitis (PSC) are the main predisposing factors in China and the U.S., respectively.⁹ Metastatic lymph nodes confer a much higher risk for tumor recurrence, but are difficult to identify during laparoscopy using white light illumination alone. New methods for image-guided surgery are needed to improve intra-operative visualization, completeness of resection, and identification of metastatic lymph nodes.

Hepatocellular carcinoma (HCC) accounts for over 840,000 deaths globally, and is emerging rapidly as a major contributor to the worldwide healthcare burden. Because few patients are diagnosed early, 5-year survival is <7%, and the median survival length is <1 year [Asrani et al., Burden of liver diseases in the world, 70 (1) (2019) 151-171.]. In the U.S., the incidence of HCC is rising steadily, and is currently growing faster than any other cancer [Ozakyol, Global Epidemiology of Hepatocellular Carcinoma (HCC Epidemiology). J Gastrointest Cancer 2017; 48:238-2407]. Conventional methods for liver imaging excel at providing anatomical features of masses. Ultrasound is recommended for patients with cirrhosis, but cannot distinguish between malignant and benign lesions. Contrast-enhanced CT and MRI detect HCC based on increased vascularity, but cannot clarify pathology for liver nodules<1-2 cm. Malignant hepatocytes uniquely overexpress targets that can be developed for improved HCC diagnosis and therapy. Thus, early detection of HCC remains a major healthcare challenge globally, and novel diagnostic options are urgently needed.

Cancer stem cells (CSCs) possess a capacity to self-renew, proliferate, and transform.^{10,11} CSCs have been postulated to serve as the origin for small duct type ICC. By comparison, large duct type ICC is believed to follow a hyperplasia-dysplasia-carcinoma in situ progression. The CSC model explains the inevitable recurrence of ICC tumors after initial therapy. EpCAM (epithelial cell adhesion molecule) expression as CSC marker is associated with aggressive behavior and poor clinical outcomes by comparison with conventional tumors. 12,13 EpCAM+ cells are normally found in bile ducts and mucous glands in large intrahepatic bile ducts.14 These tissues have been shown to harbor stem/progenitor cells that facilitate liver and bile duct regeneration. EpCAM+ progenitor cells within bile ducts are engaged in driving regenerative processes in chronic diseases that affect interlobular bile ducts. The EpCAM+ population within peribiliary glands mediate the repair of large intrahepatic bile ducts affected by primary sclerosing cholangitis and ischemia-induced cholangiopathies after orthotopic liver transplantation.^{15,16}

EpCAM is a transmembrane glycoprotein that normally functions as an epithelial-specific cell-adhesion molecule.¹⁷ This marker mediates cancer transformation via a Wnt signaling pathway.¹⁸ EpCAM is highly overexpressed in tumors of epithelial origin, such as that of intrahepatic bile ducts, and is associated with tumor progression and metastasis.¹⁹ EpCAM is an emerging CSC biomarker in ICC,²⁰⁻²² and contributes to liver regeneration.²³ These progenitor cells drive the repair process for bile duct injury from chronic inflammation. Immunohistochemistry (IHC) studies have demonstrated strong EpCAM expression in as high as 80% of ICC specimens.²⁴⁻²⁶ Furthermore, elevated levels of EpCAM expression have been found to result in low recurrence-free and overall survival.

Image-guided surgery is gaining in popularity with hepatobiliary surgeons in China.²⁷⁻²⁹ Standard laparoscopes are being adapted to collect NIR fluorescence images for use as an adjunct to conventional white light images.³⁰ These methodologies enhance image contrast to better locate tumors, identify margins, and detect metastatic lymph nodes. Surgeons currently rely on visual appearance, finger palpation, and intraoperative ultrasound to discriminate between tumor and non-tumor. These techniques are subjective, non-specific for cancer, and prone to inadequate resections and positive margins. By comparison, conventional imaging modalities, including CT, MRI, and PET, are difficult to implement for intra-operative navigation, and intraoperative ultrasound is highly operator dependent.³¹ Frozen sections for pathological evaluation obtained intraoperatively from tumor margins is time consuming and not effective for larger lesions.³² Thus, an imaging methodology that can visualize specific tumor targets with high contrast in real time may substantially improve clinical outcomes for surgical resection of ICC.

There remains a need in the art for products and methods for detecting and treating epithelial cell-derived cancers, as well as monitoring treatment of patients.

### SUMMARY

The disclosure provides peptide conjugates that specifically bind to EpCAM (herein EpCAM-specific peptide conjugates) and methods to detect and treat in patients epithelial cell-derived cancers including, but not limited to, ICC, HCC, breast cancer, colon cancer and basal cell carcinoma of the skin. EpCAM-specific peptide conjugates and methods can also be used monitor the therapeutic response of such treated patients.

The disclosure thus provides EpCAM-specific peptide conjugates. EpCAM-specific peptide conjugates can comprise the EpCAM-specific peptide HPDMFTRTHSHN (SEQ ID NO: 1), the peptide HGLHSMHNKLQD (SEQ ID NO: 2), the peptide GKPAVHYIHLRH (SEQ ID NO: 3), or the peptide HPFLHWNYGQRT (SEQ ID NO: 4). The peptide can consist of the peptide HPDMFTRTHSHN (SEQ ID NO: 1), the peptide HGLHSMHNKLQD (SEQ ID NO: 2), the peptide GKPAVHYIHLRH (SEQ ID NO: 3), or the peptide HPFLHWNYGQRT (SEQ ID NO: 4), or an analog of any of those thereof that specifically binds to EpCAM.

The disclosure provides compositions comprising a excipient (such as a pharmaceutically acceptable excipient) and an EpCAM-specific peptide conjugate.

The disclosure provides methods for detecting (including, for example, visualizing during image-guided surgery) epithelial cell-derived cancer cells such as ICC cells, HCC cells, breast cancer cells, colon cancer cells and basal cell carcinoma of the skin cells.

The disclosure provides methods for treating epithelial cell-derived cancers including, but not limited to, ICC, HCC, breast cancer, colon cancer and basal cell carcinoma of the skin.

The disclosure provides methods for monitoring the status of epithelial cell-derived cancers such as ICC, HCC, breast cancer, colon cancer and basal cell carcinoma of the skin in a patient treated with an EpCAM-specific peptide conjugate provided herein, wherein the method comprises administering the EpCAM-specific peptide conjugate comprising a detectable label to the patient to detect EpCAM expressed by the cancer cells. The detectable label can be detectable by optical, photoacoustic, ultrasound, positron emission tomography or magnetic resonance imaging.

### BRIEF DESCRIPTION OF THE DRAWINGS

This patent or application file contains at least one drawing executed in color. Copies of this patent or patent application publication with color drawing(s) will be provided by the United States Patent and Trademark Office upon request and payment of the necessary fee.

FIG. 1 shows an exemplified EpCAM-specific peptide conjugate. A) Peptide with sequence HPDMFTRTHSHN (blue) (SEQ ID NO: 1) was labeled with IRDye800 (red) via a GGGSC linker (black) (SEQ ID NO: 5) on the C-terminus to prevent steric hindrance. Peak absorbance and fluorescence emission occur at λ_{abs}=775 and λₑₘ=816 nm, respectively. B) The crystal structure of the extracellular domain (4MZV) of human EpCAM is shown. The target sequence was scrambled using this model to identify the sequence PFHDMHSNHTRT for use as control, hereafter PFH*-IRDye800.

FIG. 2 shows peptide validation with knock-down. A) HPD*-IRDye800 and anti-EpCAM-AF488 show strong binding to the surface (arrows) of siCL (control) Hep3B cells using confocal microscopy. B) The peptide and antibody show reduced intensity with siEpCAM cells. C) Quantified results show significantly greater signal for siCL versus siEpCAM with HPD* and anti-EpCAM, while PFH* showed a non-significant difference. An ANOVA was fit with terms for 3 treatments, 2 siRNA effects, and their interactions using slides in triplicate. D) Western blot shows EpCAM expression levels. E) The mean fluorescence intensity from HPD*-IRDye800 decreased significantly with competition from unlabeled HPD* in a concentration dependent manner, but not with addition of unlabeled scrambled peptide PFH*. Fluorescence intensities were measured from 10 cells chosen randomly from 3 slides. * P<0.01 by pairwise comparisons.

FIG. 3 shows peptide properties. A) HPD*-IRDye800 and anti-EpCAM-AF488 bind intensely to the surface (arrows) of Hep3B human HCC cells. A Pearson's correlation coefficient of p=0.81 was measured from the merged image. No binding was seen with the scrambled peptide PFH*-IRDye800. B) An apparent dissociation constant of k_{d}=43 nM, R²=0.99 was measured for HPD*-IRDye800 binding to Hep3B cells. The equilibrium dissociation constant k_{d}=1/kₐ was calculated by performing using a least square fit of the data to the non-linear equation I=(I₀+IₘₐₓKₐ[X])/(I₀+Kₐ[X]). I₀ and Iₘₐₓ are the initial and maximum fluorescence intensities, corresponding to no peptide and at saturation, respectively, and [X] represents the concentration of the bound peptide. Results are the mean values from 3 independent experiments. C) Peptide stability in mouse serum was measured by intravenously injecting HPD*-IRDye800 (200 μM in 200 μL of PBS) in mice (n=5). Serum was collected at various time points from 0-24 hours post-injection. The fluorescence intensity I(t) was measured and fit to a first-order kinetics model I(t)=Iₒ+A×exp(−Bt).

FIG. 4 shows the scRNAseq of ICC specimens. A) A total of 34,770 small duct cholangiocyte cells were analyzed from n=15 resected specimens of ICC, including 18,085 malignant and 16,685 paracancerous cells. Standard methods for read mapping, quality control, normalization, feature selection, dimensionality reduction, and cell clustering were applied using the Seurat toolkit. N=11 cell types were identified. EPCAM was found to be highly overexpressed in malignant cholangiocytes. B) Clusters 1 and 6 showed high expression of cancer stem cell markers THY1, PROM1, and EPCAM.

FIG. 5 shows patient-derived xenograft (PDTX) tumors. A) Representative ultrasound (US) image shows the presence of a viable human HCC tumor in mouse liver. B) White light laparoscopic image confirms presence of tumor implanted in mouse liver. IHC for EpCAM shows strong staining (arrow) for C) human HCC tumor but not D) normal mouse liver. Portal tract (central vein, hepatic artery, and bile ducts) is identified (arrow) on pathology.

FIG. 6 shows in vivo imaging of orthotopic HCC tumors. A) Representative NIR fluorescence images collected in vivo are shown from orthotopically implanted human Hep3B (EpCAM+) HCC xenograft tumors in immunocompromised mice at 1.5 hours following systemic administration of HPD*-IRDye800 and PFH*-IRDye800 (150 UM, 200 μL). B) The NIR fluorescence intensity for HPD*-IRDye800 was significantly greater than that for PFH*-IRDye800, *P<1×10-4 by paired t-test. C) The T/B ratio was measured from tumor from 0-48 hours post-injection of ALL*-IRDye800, FEA*-IRDye800 (150 UM, 200 μL), ALL* (block, 1.5 mM, 100 μL), and ICG (2.46 mg/kg). A total of n=8 animals were studied per group. Quantified intensities shown peak uptake of ALL*-IRDye800 at 1.5 hours post-injection with near complete clearance by ~48 hours. The mean (±SD) T/B ratio for ALL*-IRDye800 was significantly greater than that for FEA*-IRDye800, ALL* (block), ICG, and GPC3− with an increase of 1.7, 2.1, 1.4 and 1.9-fold increase, respectively, at 1.5 hours post-injection. P-values were calculated using a one-way ANOVA model. Background was defined as the adjacent non-tumor region with equal area of the tumor region.

FIG. 7 shows in vivo laparoscopic imaging of orthotopic tumors. Representative A,C) white light (WL) and B,D) NIR fluorescence (FL) images collected in vivo are shown at 1.5 hours post-injection of HPD*-IRDye800. A similar set of images were collected using PFH*-IRDye800. E) Quantified fluorescence intensities show a significantly greater mean (±SD) value for the T/B ratio of HPD*-IRDye800 versus that for PFH*-IRDye800 with 1.7-fold increase. P-values were calculated using a paired t-test. Background was defined as the adjacent non-tumor region with equal area of the tumor region.

FIG. 8 shows specific binding to tumor ex vivo. A) Increased anti-EpCAM reactivity shows presence of EpCAM expression by human HCC tumor (arrow) present next to normal mouse liver. Bright fluorescence is seen from binding of B) HPD*-IRDye800 and C) anti-EpCAM-488 to the cell surface (arrow) of HCC tumor but not to normal mouse liver. D) Merged image shows co-localization of EpCAM-specific peptide and antibody binding.

FIG. 9 shows validation of EpCAM expression. In a total of n=57 ICC specimens, A) none (0+), B) minimal (1+), C) moderate (2+), and D) strong (3+) reactivity was found in 4 (7%), 8 (14%), 24 (42%), and 21 (37%) of ICC specimens ex vivo using immunohistochemistry (IHC). A standard scoring system was used: 0+, no reactivity or membrane staining in <10% of tumor cells; 1+, a faint/barely perceptible membrane staining is detected in >10% of tumor cells. Cells exhibit incomplete membrane staining; 2+, a weak to moderate complete membrane staining is observed in >10% of tumor cells; 3+, a strong complete membrane staining is observed in >10% of tumor cells.

FIG. 10 shows specific peptide binding to human liver cancer ex vivo. A) HPD*-IRDye800 (red) and B) anti-EpCAM-AF488 (green) show strong staining to the surface of HCC cells (arrows) and substantially reduced staining to normal human liver is seen using immunofluorescence. C) Quantified results show a 2.6-fold increase in mean fluorescence intensity for the EpCAM-specific peptide to distinguish HCC (n=36) from normal (n=12) human specimens, P<1×10⁻⁵ by unpaired t-test. ROC curves show 85% sensitivity and 66% specificity with AUC=0.81 to distinguish HCC from normal.

FIG. 11 shows peptide biodistribution. NIR fluorescence images are shown as an overlay on white light images from major organs at 1.5 hours post-injection of A) HPD*-IRDye800, B) PFH*-IRDye800, C) HPD* (block), and D) ICG. E) Quantified results showed uptake of HPD*-IRDye800 by the PDX HCC tumor was significantly higher than that for the other groups.

FIG. 12 shows animal necropsy. Mice were sacrificed at XX hours post-injection with HPD*-IRDye800 (XSX mM, XXX mL). No signs of acute toxicity are seen on histology (H&E) of vital organs, including A) brain, B) heart, C) liver, D) spleen, E) lung, F) kidney, G) stomach, and H) colon.

FIG. 13 shows in vivo whole-body fluorescence imaging. A) Images were collected with excitation at λₑₓ=800 nm before (pre) and at 0, 0.5, 1.0, 1.25, 1.5, 2, 4 and 24 hours post-injection of HPD*-IRDye800. Peak T/B ratio from the tumor site (circle) was observed at 1.5 hours. PFH*-IRDye800 was administered for use as control. Unlabeled HPD* was injected 20 min prior to HPD*-IRDye800 to compete for binding (block). These controls showed reduced values over 24 hours. ICG (control) showed non-specific uptake. B) The quantified T/B ratio confirms a peak uptake of HPD*-IRDye800 by tumor at 1.5 hours. The adjacent non-tumor tissue region with equal area to the tumor region was used for background. The quantified T/B ratio for WKG*-IRDye800 was significantly greater than those of HPD*-IRDye800, block, and ICG. C) A half-life of T_{1/2}=2.6 hours was measured in mouse serum for HPD*-IRDye800.

FIG. 14 shows micrometastases detected. A) White light (WL) and B) fluorescence (FL) images of liver were collected in vivo laparoscopically at 1.5 hours post-injection of HPD*-IRDye800. Primary human HCC PDX tumor (arrow) is shown surrounded by foci of micrometastases (arrowheads). C) Corresponding histology (H&E) confirms HCC tumor (arrow). D) Human-specific anti-cytokeratin showed presence of primary HCC PDX tumor (arrow) and nearby foci of micrometastases (arrowhead). E) Antibody staining confirmed EpCAM expression in primary tumor (arrow) and micrometastases (arrowhead). F) Expanded view of black box from panel E) is shown.

FIG. 15 shows liver micrometastases. A) HPD*-IRDye800 (cyan) and B) anti-EpCAM-AF488 (green) showed strong binding to foci of HCC micrometastases (arrow) in liver. C) Merged image shows co-binding of peptide and antibody. D) Anti-cytokeratin stain confirms presence of human tumor (arrow). E) DAPI stain shows nuclei. F) Corresponding histology (H&E) verifies HCC tumor in liver.

FIG. 16 shows lung micrometastases. A) HPD*-IRDye800 (cyan) and B) anti-EpCAM-AF488 (green) showed strong binding to foci of HCC micrometastases (arrow) in lung. C) Merged image shows co-binding of peptide and antibody. D) Anti-cytokeratin stain confirms presence of human tumor (arrow). E) DAPI stain shows nuclei. F) Corresponding histology (H&E) verifies HCC tumor in lung.

### DETAILED DESCRIPTION

### Peptides and Conjugates

The disclosure provides EpCAM-specific peptide conjugates comprising the EpCAM-specific peptide HPDMFTRTHSHN (SEQ ID NO: 1), HGLHSMHNKLQD (SEQ ID NO: 2), GKPAVHYIHLRH (SEQ ID NO: 3), or HPFLHWNYGQRT (SEQ ID NO: 4). The EpCAM-specific peptide can consist of the peptide HGLHSMHNKLQD (SEQ ID NO: 2), the peptide GKPAVHYIHLRH (SEQ ID NO: 3), or the peptide HPFLHWNYGQRT (SEQ ID NO: 4). The disclosure also provides analogs of the EpCAM-specific peptides. Also contemplated by the present disclosure are peptides that compete with peptides provided herein for binding to EpCAM.

The EpCAM-specific peptide conjugates can have one or more of the following properties: the peptide can have an Eₜ<−300 in a structural model as described in Example 2, the conjugate can attain a >3-fold reduction in fluorescence intensity using siRNA knockdown, the conjugate can attain a >5-fold reduction in fluorescence intensity using competition with unlabeled peptides, the conjugate can attain a calculated ρ>0.8 for in vitro co-localization of optimized peptide and antibody binding to cells in vitro, the conjugate can have an apparent dissociation constant (binding affinity) for EpCAM of k_{d}<40 nM, and the conjugate can have a serum half-life of T_{1/2}>5 hours.

The disclosure provides peptide conjugates comprising an EpCAM-specific peptide provided herein. A “peptide conjugate” comprises at least two components, a peptide provided herein and another moiety attached to the peptide. In the EpCAM-specific peptide conjugates provided herein, the only component of the peptide conjugate that contributes to EpCAM binding is the EpCAM-specific peptide. In other words, an EpCAM-specific peptide conjugate “consists essentially of” an EpCAM-specific peptide provided herein. The other moiety can comprise amino acids, but the EpCAM-specific peptide is not linked to those amino acids in nature and the other amino acids do not affect alter the efficacy of the EpCAM-specific peptide in EpCAM binding. For example, an EpCAM-specific peptide conjugate can comprise amino acids that impart cell permeability to the EpCAM-specific peptide, such as HIV TAT amino acids GRKKRRQRRRPQ (SEQ ID NO: 5). The other amino acids can be linked to the peptides provided herein by typical peptide bonds or by other linkages known in the art. Moreover, the other moiety in a conjugate contemplated herein is not a phage in a phage display library or a component of any other type of peptide display library.

The disclosure provides methods for detecting epithelial cell-derived cancers such as ICC, HCC, breast cancer, colon cancer and basal cell carcinoma of the skin, and methods for monitoring the status of epithelial cell-derived cancers such as ICC, HCC, breast cancer, colon cancer and basal cell carcinoma of the skin in a patient treated with an EpCAM-specific peptide conjugate provided herein, which methods comprise administration of an EpCAM-specific peptide conjugate to a patient to detect EpCAM expressed on the surface of cancer cells. The EpCAM-specific peptide conjugate comprises a detectable label that is detected in the methods.

A peptide conjugate can comprise at least one detectable label as a moiety attached to a peptide provided herein. The detectable label can be detected, for example, by optical, ultrasound, PET, SPECT, or magnetic resonance imaging. The label detectable by optical imaging can be, for example, fluorescein isothiocyanate (FITC), Cy5, Cy5.5 or IRdye800 (also known as IR800CW). The label detectable by magnetic resonance imaging can be, for example, gadolinium, Gd-DOTA or an iron oxide nanoparticle. More detectable labels contemplated are set out below.

A detectable label can be attached to a peptide provided herein by a peptide linker. The terminal amino acid of the linker can be a lysine such as in the exemplary linker GGGSK (SEQ ID NO: 6).

A peptide conjugate can comprise at least one therapeutic moiety attached to a peptide provided herein. The therapeutic moiety can be a chemopreventative or chemotherapeutic agent. For example, the chemopreventative agent can be celecoxib. As other non-limiting examples, the chemotherapeutic agent can be carboplatin, paclitaxel, cisplatin, 5-fluorouracil (5-FU), oxaliplatin, capecitabine, chloambucil, sorafenib or irinotecan. The therapeutic moiety can be a nanoparticle or micelle encapsulating another therapeutic moiety. For example, carboplatin, paclitaxel, cisplatin, 5-fluorouracil (5-FU), oxaliplatin, capecitabine, chloambucil, sorafenib or irinotecan can be encapsulated. More therapeutic moieties contemplated are set out below.

A peptide conjugate can comprise at least one detectable label attached to the peptide or multimer form of the peptide, and at least one therapeutic moiety attached to the peptide or multimer form of the peptide.

### Linkers, Peptides and Peptide Analogs

As used herein, a “linker” is a sequence of amino acids located at the C-terminus of a peptide of the disclosure. The linker sequence can terminate with a lysine residue.

The presence of a linker can result in at least a 1% increase in detectable binding of an EpCAM-specific peptide conjugate provided herein to cells compared to the detectable binding of the peptide conjugate in the absence of the linker. The increase in detectable binding can be at least 2%, at least 3%, at least 4%, at least 5%, at least 6%, at least 7%, at least 8%, at least 9%, at least 10%, at least 11%, at least 12%, at least 13%, at least 14%, at least 15%, at least 16%, at least 17%, at least 18%, at least 19%, at least 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 99%, at least about 2-fold, at least about 3-fold, at least about 4-fold, at least about 5-fold, at least about 6-fold, at least about 7-fold, at least about 8-fold, at least about 9-fold, at least about 10-fold, at least about 15-fold, at least about 20-fold, at least about 25-fold, at least about 30-fold, at least about 35-fold, at least about 40-fold, at least about 45-fold, at least about 50-fold, at least about 100-fold or more.

The term “peptide” refers to molecules of 2 to 50 amino acids, molecules of 3 to 20 amino acids, and those of 6 to 15 amino acids. Peptides and linkers contemplated herein can be 5 amino acids in length. A polypeptide or linker can be 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 or more amino acids in length.

The peptide of a peptide conjugate provided herein can be presented in multimer form. Various scaffolds are known in the art upon which multiple peptides can be presented. A peptide can be presented in multimer form on a trilysine dendritic wedge. A peptide can be presented in dimer form using an aminohexanoic acid linker. Other scaffolds known in the art include, but are not limited to, other dendrimers and polymeric (e.g., PEG) scaffolds.

It will be understood that peptides and linkers provided herein optionally incorporate modifications known in the art and that the location and number of such modifications are varied to achieve an optimal effect in the peptide and/or linker analog.

A peptide analog having a structure based on one of the peptides disclosed herein (the “parent peptide”) can differ from the parent peptide in one or more respects. Accordingly, as appreciated by one of ordinary skill in the art, the teachings regarding the parent peptides provided herein can also be applicable to the peptide analogs.

A peptide analog can comprise one or more D amino acids to increase the resistance of the peptides to proteases to increase serum stability.

The peptide analog can comprise the structure of a parent peptide, except that the peptide analog comprises one or more non-peptide bonds in place of peptide bond(s). The peptide analog can comprise in place of a peptide bond, an ester bond, an ether bond, a thioether bond, an amide bond, and the like. The peptide analog can be a depsipeptide comprising an ester linkage in place of a peptide bond.

The peptide analog can comprise the structure of a parent peptide described herein, except that the peptide analog comprises one, two, three, four or more amino acid substitutions, e.g., one, two, three, four or more conservative amino acid substitutions. Conservative amino acid substitutions are known in the art, and include amino acid substitutions in which one amino acid having certain physical and/or chemical properties is exchanged for another amino acid that has the same chemical or physical properties. For instance, the conservative amino acid substitution can be an acidic amino acid substituted for another acidic amino acid (e.g., Asp or Glu), an amino acid with a nonpolar side chain substituted for another amino acid with a nonpolar side chain (e.g., Ala, Gly, Val, Ile, Leu, Met, Phe, Pro, Trp, Val, etc.), a basic amino acid substituted for another basic amino acid (Lys, Arg, etc.), an amino acid with a polar side chain substituted for another amino acid with a polar side chain (Asn, Cys, Gln, Ser, Thr, Tyr, etc.), etc.

The peptide analog can comprise one, two, three, four or more synthetic amino acids, e.g., an amino acid non-native to a mammal. Synthetic amino acids include β-alanine (β-Ala), N-□-methyl-alanine (Me-Ala), aminobutyric acid (Abu), γ-aminobutyric acid (γ-Abu), aminohexanoic acid (ε-Ahx), aminoisobutyric acid (Aib), aminomethylpyrrole carboxylic acid, aminopiperidinecarboxylic acid, aminoserine (Ams), aminotetrahydropyran-4-carboxylic acid, arginine N-methoxy-N-methyl amide, β-aspartic acid (β-Asp), azetidine carboxylic acid, 3-(2-benzothiazolyl) alanine, α-tert-butylglycine, 2-amino-5-ureido-n-valeric acid (citrulline, Cit), β-Cyclohexylalanine (Cha), acetamidomethyl-cysteine, diaminobutanoic acid (Dab), diaminopropionic acid (Dpr), dihydroxyphenylalanine (DOPA), dimethylthiazolidine (DMTA), γ-Glutamic acid (γ Glu), homoserine (Hse), hydroxyproline (Hyp), isoleucine N-methoxy-N-methyl amide, methyl-isoleucine (Melle), isonipecotic acid (Isn), methyl-leucine (MeLeu), methyl-lysine, dimethyl-lysine, trimethyl-lysine, methanoproline, methionine-sulfoxide (Met(O)), methionine-sulfone (Met (O₂)), norleucine (Nle), methyl-norleucine (Me-Nle), norvaline (Nva), ornithine (Orn), para-aminobenzoic acid (PABA), penicillamine (Pen), methylphenylalanine (MePhe), 4-Chlorophenylalanine (Phe(4-CI)), 4-fluorophenylalanine (Phe(4-F)), 4-nitrophenylalanine (Phe(4-NO2)), 4-cyanophenylalanine ((Phe(4-CN)), phenylglycine (Phg), piperidinylalanine, piperidinylglycine, 3,4-dehydroproline, pyrrolidinylalanine, sarcosine (Sar), selenocysteine (Sec), O-Benzyl-phosphoserine, 4-amino-3-hydroxy-6-methylheptanoic acid (Sta), 4-amino-5-cyclohexyl-3-hydroxypentanoic acid (ACHPA), 4-amino-3-hydroxy-5-phenylpentanoic acid (AHPPA), 1,2,3,4,-tetrahydro-isoquinoline-3-carboxylic acid (Tic), tetrahydropyranglycine, thienylalanine (Thi), O-benzyl-phosphotyrosine, O-Phosphotyrosine, methoxytyrosine, ethoxytyrosine, O-(bis-dimethylamino-phosphono)-tyrosine, tyrosine sulfate tetrabutylamine, methyl-valine (MeVal), and alkylated 3-mercaptopropionic acid.

The peptide analog can comprise one, two, three, four or more non-conservative amino acid substitutions and the peptide analog still functions to a similar extent, the same extent, or an improved extent as the parent peptide. The peptide analog can comprise one or more non-conservative amino acid substitutions exhibits about the same or greater binding to HCC cells in comparison to the parent peptide.

The peptide analog can comprise one, two, three, four or more amino acid insertions or deletions, in comparison to the parent peptide described herein. The peptide analog can comprise an insertion of one or more amino acids in comparison to the parent peptide. The peptide analog can comprise a deletion of one or more amino acids in comparison to the parent peptide. The peptide analog can comprise an insertion of one or more amino acids at the N- or C-terminus in comparison to the parent peptide. The peptide analog can comprise a deletion of one or more amino acids at the N- or C-terminus in comparison to the parent peptide.

Peptide analogs provided can exhibit about the same or greater binding to EpCAM as the original peptide.

The peptides and peptide analogs provided herein can be PEGylated or acetylated to improve serum stability.

### Detectable Labels

As used herein, a “detectable label” is any label that can be used to identify the binding of a composition of the disclosure to HCC cells. Non-limiting examples of detectable labels are fluorophores, chemical or protein tags that enable the visualization of a polypeptide. Visualization in certain aspects is carried out with the naked eye, or a device (for example and without limitation, an endoscope) and can also involve an alternate light or energy source.

Fluorophores, chemical and protein tags that are contemplated for use herein include, but are not limited to, FITC, Cy5, Cy 5.5, Cy 7, Li-Cor, a radiolabel, biotin, luciferase, 1,8-ANS (1-Anilinonaphthalene-8-sulfonic acid), 1-Anilinonaphthalene-8-sulfonic acid (1,8-ANS), 5-(and-6)-Carboxy-2′,7′-dichlorofluorescein pH 9.0, 5-FAM pH 9.0, 5-ROX (5-Carboxy-X-rhodamine, triethylammonium salt), 5-ROX pH 7.0, 5-TAMRA, 5-TAMRA PH 7.0, 5-TAMRA-MeOH, 6 JOE, 6,8-Difluoro-7-hydroxy-4-methylcoumarin pH 9.0, 6-Carboxyrhodamine 6G pH 7.0, 6-Carboxyrhodamine 6G, hydrochloride, 6-HEX, SE pH 9.0, 6-TET, SE pH 9.0, 7-Amino-4-methylcoumarin pH 7.0, 7-Hydroxy-4-methylcoumarin, 7-Hydroxy-4-methylcoumarin pH 9.0, Alexa 350, Alexa 405, Alexa 430, Alexa 488, Alexa 532, Alexa 546, Alexa 555, Alexa 568, Alexa 594, Alexa 647, Alexa 660, Alexa 680, Alexa 700, Alexa Fluor 430 antibody conjugate pH 7.2, Alexa Fluor 488 antibody conjugate pH 8.0, Alexa Fluor 488 hydrazide-water, Alexa Fluor 532 antibody conjugate pH 7.2, Alexa Fluor 555 antibody conjugate pH 7.2, Alexa Fluor 568 antibody conjugate pH 7.2, Alexa Fluor 610 R-phycoerythrin streptavidin pH 7.2, Alexa Fluor 647 antibody conjugate pH 7.2, Alexa Fluor 647 R-phycoerythrin streptavidin pH 7.2, Alexa Fluor 660 antibody conjugate pH 7.2, Alexa Fluor 680 antibody conjugate pH 7.2, Alexa Fluor 700 antibody conjugate pH 7.2, Allophycocyanin pH 7.5, AMCA conjugate, Amino Coumarin, APC (allophycocyanin), Atto 647, BCECF pH 5.5, BCECF pH 9.0, BFP (Blue Fluorescent Protein), Calcein, Calcein pH 9.0, Calcium Crimson, Calcium Crimson Ca2+, Calcium Green, Calcium Green-1 Ca2+, Calcium Orange, Calcium Orange Ca2+, Carboxynaphthofluorescein pH 10.0, Cascade Blue, Cascade Blue BSA pH 7.0, Cascade Yellow, Cascade Yellow antibody conjugate pH 8.0, CFDA, CFP (Cyan Fluorescent Protein), CI-NERF pH 2.5, CI-NERF pH 6.0, Citrine, Coumarin, Cy 2, Cy 3, Cy 3.5, Cy 5, C5.5, CyQUANT GR-DNA, Dansyl Cadaverine, Dansyl Cadaverine, MeOH, DAPI, DAPI-DNA, Dapoxyl(2-aminoethyl) sulfonamide, DDAO pH 9.0, Di-8 ANEPPS, Di-8-ANEPPS-lipid, Dil, DiO, DM-NERF pH 4.0, DM-NERF pH 7.0, DsRed, DTAF, dTomato, eCFP (Enhanced Cyan Fluorescent Protein), eGFP (Enhanced Green Fluorescent Protein), Eosin, Eosin antibody conjugate pH 8.0, Erythrosin-5-isothiocyanate pH 9.0, eYFP (Enhanced Yellow Fluorescent Protein), FDA, FITC antibody conjugate pH 8.0, FIASH, Fluo-3, Fluo-3 Ca2+, Fluo-4, Fluor-Ruby, Fluorescein, Fluorescein 0.1 M NaOH, Fluorescein antibody conjugate pH 8.0, Fluorescein dextran pH 8.0, Fluorescein pH 9.0, Fluoro-Emerald, FM 1-43, FM 1-43 lipid, FM 4-64, FM 4-64, 2% CHAPS, Fura Red Ca2+, Fura Red, high Ca, Fura Red, low Ca, Fura-2 Ca2+, Fura-2, Fura-2, GFP (S65T), HcRed, Indo-1 Ca2+, Indo-1, Ca free, Indo-1, Ca saturated, IDRdye800 (IR800CW), JC-1, JC-1 pH 8.2, Lissamine rhodamine, Lucifer Yellow, CH, Magnesium Green, Magnesium Green Mg2+, Magnesium Orange, Marina Blue, mBanana, mCherry, mHoneydew, mOrange, mPlum, mRFP, mStrawberry, mTangerine, NBD-X, NBD-X, MeOH, NeuroTrace 500/525, green fluorescent Nissl stain-RNA, Nile Blue, Nile Red, Nile Red-lipid, Nissl, Oregon Green 488, Oregon Green 488 antibody conjugate pH 8.0, Oregon Green 514, Oregon Green 514 antibody conjugate pH 8.0, Pacific Blue, Pacific Blue antibody conjugate pH 8.0, Phycoerythrin, R-Phycoerythrin pH 7.5, ReAsH, Resorufin, Resorufin pH 9.0, Rhod-2, Rhod-2 Ca2⁺, Rhodamine, Rhodamine 110, Rhodamine 110 pH 7.0, Rhodamine 123, MeOH, Rhodamine Green, Rhodamine phalloidin pH 7.0, Rhodamine Red-X antibody conjugate pH 8.0, Rhodamine Green pH 7.0, Rhodol Green antibody conjugate pH 8.0, Sapphire, SBFI-Na⁺, Sodium Green Na⁺, Sulforhodamine 101, Tetramethylrhodamine antibody conjugate pH 8.0, Tetramethylrhodamine dextran pH 7.0, and Texas Red-X antibody conjugate pH 7.2.

Non-limiting examples of chemical tags contemplated herein include radiolabels. For example and without limitation, radiolabels that contemplated in the compositions and methods of the present disclosure include ¹¹C, ¹³N, ¹⁵O, ¹⁸F, ³²P, ⁵²Fe, ⁶²Cu, ⁶⁴Cu, ⁶⁷Cu, ⁶⁷Ga, ⁶⁸Ga, ⁸⁶Y, ⁸⁹Zr, ⁹⁰Y, ⁹⁴mTc, ⁹⁴Tc, ⁹⁵Tc, ⁹⁹mTc, ¹⁰³Pd, ¹⁰⁵Rh, ¹⁰⁹Pd, ¹¹Ag, ¹¹In, ¹²³I, ¹²⁴I, ¹²⁵I, ¹³¹I, ¹⁴⁰La, ¹⁴⁹Pm, ¹⁵³Sm, ¹⁵⁴⁻¹⁵⁹Gd, ¹⁶⁵Dy, ¹⁶⁶Dy, ¹⁶⁶Ho, ¹⁶⁹Yb, ¹⁷⁵Yb, ¹⁷⁵Lu, ¹⁷⁷Lu, ¹⁸⁶Re, ¹⁸⁸Re, ¹⁹²Ir, ¹⁹⁸Au, ¹⁹⁹Au, and ²¹²Bi.

For magnetic resonance imaging, non-limiting examples of detectable labels contemplated herein are gadolinium (Gd), Gd-DOTA and iron oxide nanoparticles.

For positron emission tomography (PET) tracers including, but not limited to, carbon-11, nitrogen-13, oxygen-15 and fluorine-18 are used.

A worker of ordinary skill in the art will appreciate that there are many such detectable labels that can be used to visualize a cell, in vitro, in vitro or ex vivo.

### Therapeutic Moieties

Therapeutic moieties contemplated herein include, but are not limited to, polypeptides (including protein therapeutics) or peptides, small molecules, chemotherapeutic agents, or combinations thereof.

The term “small molecule”, as used herein, refers to a chemical compound, for instance a peptidometic or oligonucleotide that can optionally be derivatized, or any other low molecular weight organic compound, either natural or synthetic.

By “low molecular weight” is meant compounds having a molecular weight of less than 1000 Daltons, typically between 300 and 700 Daltons. Low molecular weight compounds, in various aspects, are about 100, about 150, about 200, about 250, about 300, about 350, about 400, about 450, about 500, about 550, about 600, about 650, about 700, about 750, about 800, about 850, about 900, about 1000 or more Daltons.

The therapeutic moiety can be a protein therapeutic. Protein therapeutics include, without limitation, cellular or circulating proteins as well as fragments and derivatives thereof. Still other therapeutic moieties include polynucleotides, including without limitation, protein coding polynucleotides, polynucleotides encoding regulatory polynucleotides, and/or polynucleotides which are regulatory in themselves. Optionally, the compositions comprise a combination of the compounds described herein.

Protein therapeutics can include cytokines or hematopoietic factors including without limitation IL-1 alpha, IL-1 beta, IL-2, IL-3, IL-4, IL-5, IL-6, IL-11, colony stimulating factor-1 (CSF-1), M-CSF, SCF, GM-CSF, granulocyte colony stimulating factor (G-CSF), EPO, interferon-alpha (IFN-alpha), consensus interferon, IFN-beta, IFN-gamma, IL-7, IL-8, IL-9, IL-10, IL-12, IL-13, IL-14, IL-15, IL-16, IL-17, IL-18, thrombopoietin (TPO), angiopoietins, for example Ang-1, Ang-2, Ang-4, Ang-Y, the human angiopoietin-like polypeptide, vascular endothelial growth factor (VEGF), angiogenin, bone morphogenic protein-1, bone morphogenic protein-2, bone morphogenic protein-3, bone morphogenic protein-4, bone morphogenic protein-5, bone morphogenic protein-6, bone morphogenic protein-7, bone morphogenic protein-8, bone morphogenic protein-9, bone morphogenic protein-10, bone morphogenic protein-11, bone morphogenic protein-12, bone morphogenic protein-13, bone morphogenic protein-14, bone morphogenic protein-15, bone morphogenic protein receptor IA, bone morphogenic protein receptor IB, brain derived neurotrophic factor, ciliary neutrophic factor, ciliary neutrophic factor receptor, cytokine-induced neutrophil chemotactic factor 1, cytokine-induced neutrophil, chemotactic factor 2a, cytokine-induced neutrophil chemotactic factor 2B, B endothelial cell growth factor, endothelin 1, epidermal growth factor, epithelial-derived neutrophil attractant, fibroblast growth factor 4, fibroblast growth factor 5, fibroblast growth factor 6, fibroblast growth factor 7, fibroblast growth factor 8, fibroblast growth factor 8b, fibroblast growth factor 8c, fibroblast growth factor 9, fibroblast growth factor 10, fibroblast growth factor acidic, fibroblast growth factor basic, glial cell line-derived neutrophic factor receptor α1, glial cell line-derived neutrophic factor receptor α2, growth related protein, growth related protein α, growth related protein β, growth related protein γ, heparin binding epidermal growth factor, hepatocyte growth factor, hepatocyte growth factor receptor, insulin-like growth factor I, insulin-like growth factor receptor, insulin-like growth factor II, insulin-like growth factor binding protein, keratinocyte growth factor, leukemia inhibitory factor, leukemia inhibitory factor receptor α, nerve growth factor nerve growth factor receptor, neurotrophin-3, neurotrophin-4, placenta growth factor, placenta growth factor 2, platelet-derived endothelial cell growth factor, platelet derived growth factor, platelet derived growth factor A chain, platelet derived growth factor AA, platelet derived growth factor AB, platelet derived growth factor B chain, platelet derived growth factor BB, platelet derived growth factor receptor α, platelet derived growth factor receptor β, pre-B cell growth stimulating factor, stem cell factor receptor, TNF, including TNF0, TNF1, TNF2, transforming growth factor α, transforming growth factor β, transforming growth factor β1, transforming growth factor β1.2, transforming growth factor β2, transforming growth factor 33, transforming growth factor β5, latent transforming growth factor β1, transforming growth factor β binding protein I, transforming growth factor β binding protein II, transforming growth factor β binding protein III, tumor necrosis factor receptor type I, tumor necrosis factor receptor type II, urokinase-type plasminogen activator receptor, vascular endothelial growth factor, and chimeric proteins and biologically or immunologically active fragments thereof.

Therapeutic moieties can also include chemotherapeutic agents. A chemotherapeutic agent contemplated for use in a peptide conjugate provided herein includes, without limitation, alkylating agents including: nitrogen mustards, such as mechlor-ethamine, cyclophosphamide, ifosfamide, melphalan and chlorambucil; nitrosoureas, such as carmustine (BCNU), lomustine (CCNU), and semustine (methyl-CCNU); ethylenimines/methylmelamine such as thriethylenemelamine (TEM), triethylene, thiophosphoramide (thiotepa), hexamethylmelamine (HMM, altretamine); alkyl sulfonates such as busulfan; triazines such as dacarbazine (DTIC); antimetabolites including folic acid analogs such as methotrexate and trimetrexate, pyrimidine analogs such as 5-fluorouracil, capecitabine, fluorodeoxyuridine, gemcitabine, cytosine arabinoside (AraC, cytarabine), 5-azacytidine, 2,2′-difluorodeoxycytidine, purine analogs such as 6-mercaptopurine, 6-thioguanine, azathioprine, 2′-deoxycoformycin (pentostatin), erythrohydroxynonyladenine (EHNA), fludarabine phosphate, and 2-chlorodeoxyadenosine (cladribine, 2-CdA); natural conjugates including antimitotic drugs such as paclitaxel, vinca alkaloids including vinblastine (VLB), vincristine, and vinorelbine, taxotere, estramustine, and estramustine phosphate; epipodophylotoxins such as etoposide and teniposide; antibiotics such as actimomycin D, daunomycin (rubidomycin), doxorubicin, mitoxantrone, idarubicin, bleomycins, plicamycin (mithramycin), mitomycinC, and actinomycin; enzymes such as L-asparaginase; biological response modifiers such as interferon-alpha, IL-2, G-CSF and GM-CSF; miscellaneous agents including platinium coordination complexes such as oxaliplatin, cisplatin and carboplatin, anthracenediones such as mitoxantrone, substituted urea such as hydroxyurea, methylhydrazine derivatives including N-methylhydrazine (MIH) and procarbazine, adrenocortical suppressants such as mitotane (o,p′-DDD) and aminoglutethimide; hormones and antagonists including adrenocorticosteroid antagonists such as prednisone and equivalents, dexamethasone and aminoglutethimide; topoisomerase inhibitors such as irinotecan; progestin such as hydroxyprogesterone caproate, medroxyprogesterone acetate and megestrol acetate; estrogen such as diethylstilbestrol and ethinyl estradiol equivalents; antiestrogen such as tamoxifen; androgens including testosterone propionate and fluoxymesterone/equivalents; antiandrogens such as flutamide, gonadotropin-releasing hormone analogs and leuprolide; and non-steroidal antiandrogens such as flutamide. Chemotherapeutic agents such as gefitinib, sorafenib and erlotinib are also specifically contemplated.

Therapeutic moieties to be attached to a peptide described herein also include nanoparticles or micelles that, in turn, encapsulate another therapeutic moiety. The nanoparticles can be polymeric nanoparticles such as described in Zhang et al., ACS NANO, 2 (8): 1696-1709 (2008) or Zhong et al., Biomacromolecules, 15:1955-1969 (2014). The micelles can be polymeric micelles such as octadecyl lithocholate micelles described in Khondee et al., J. Controlled Release, 199:114-121 (2015) and WO 2017/096076 (published Jun. 8, 2017). The peptide conjugates comprising nanoparticles or micelles can encapsulate, for example, carboplatin, paclitaxel, cisplatin, 5-fluorouracil (5-FU), oxaliplatin, capecitabine or irinotecan.

### Compositions

The disclosure provides a composition comprising at least one peptide or peptide conjugate provided herein and a pharmaceutically acceptable excipient.

### Methods

The disclosure provides methods for specifically detecting epithelial cell-derived cancers cells such as ICC cells, HCC cells, breast cancer cells, colon cancer cells and basal cell carcinoma of the skin cells in a patient comprising the steps of administering an EpCAM-specific peptide conjugate provided herein comprising a detectable label to the patient and detecting binding of the EpCAM-specific peptide conjugate to the cells. Such methods can be used, for example, to determine the presence of epithelial cell-derived cancers in a patient. Another example of use of such methods is visualizing epithelial cell-derived cancer cells during image-guided surgery.

Methods provided herein can comprise the acquisition of a tissue sample from a patient. The tissue sample can be a tissue or organ of said patient.

The disclosure provides a method for delivering a therapeutic moiety to a patient comprising the step of administering a peptide conjugate provided herein comprising the therapeutic moiety to the patient.

The disclosure provides a method for treating an epithelial cell-derived cancer (such as ICC, HCC, breast cancer, colon cancer and basal cell carcinoma of the skin) in a patient comprising the step of administering an EpCAM-specific peptide conjugate provided herein comprising a therapeutic moiety to the patient.

The disclosure provides a method of determining the effectiveness of a treatment for epithelial cell-derived cancers (such as ICC, HCC, breast cancer, colon cancer and basal cell carcinoma of the skin) in a patient comprising the step of administering an EpCAM-specific peptide conjugate provided herein comprising a detectable label to the patient, visualizing a first amount of cells labeled with the peptide conjugate, and comparing the first amount to a previously-visualized second amount of cells labeled with the peptide conjugate, wherein a decrease in the first amount cells labeled relative to the previously-visualized second amount of cells labeled is indicative of effective treatment. A decrease of 5% can be indicative of effective treatment. A decrease of about 10%, about 15%, about 20%, about 25%, about 30%, about 35%, about 40%, about 45%, about 50%, about 55%, about 60%, about 65%, about 70%, about 75%, about 80%, about 85%, about 90%, about 95% or more can indicative of effective treatment. The method can further comprise obtaining a biopsy of the cells labeled by the peptide conjugate.

Methods provided herein can be used for primary, secondary or recurrent cancers.

The phrases “specific for,” “specifically binds to” or “specifically detects” mean that the peptide conjugate binds to and is detected in association with EpCAM on a cell, and the conjugate does not bind to and is not detected in association with another protein on the cell at the level of sensitivity at which the method is carried out.

Peptides or peptide conjugates and compositions thereof provided herein can be delivered by any route that effectively reaches target cells (e.g., cancer cells) in a patient including, but not limited to, administration by an intravenous, topical, oral or nasal route.

The disclosure provides a kit for administering a composition provided herein to a patient in need thereof, where the kit comprises a composition provided herein, instructions for use of the composition and a device for administering the composition to the patient.

### Dosages

Dosages of a peptide or peptide conjugate provided herein are administered as a dose measured in, for example, mg/kg. Contemplated mg/kg doses include, but are not limited to, about 1 mg/kg to about 60 mg/kg. Illustrative specific ranges of doses in mg/kg include about 1 mg/kg to about 20 mg/kg, about 5 mg/kg to about 20 mg/kg, about 10 mg/kg to about 20 mg/kg, about 25 mg/kg to about 50 mg/kg, and about 30 mg/kg to about 60 mg/kg. The precise effective amount for a subject will depend upon the subject's body weight, size, and health; the nature and extent of the condition; and the therapeutic or combination of therapeutics selected for administration. Therapeutically effective amounts for a given situation can be determined by routine experimentation that is within the skill and judgment of the clinician.

“Effective amount” as used herein refers to an amount of a peptide or peptide conjugate provided herein sufficient to visualize the identified disease or condition, or to exhibit a detectable therapeutic effect. That is, the effect is detected by, for example, an improvement in clinical condition or reduction in symptoms. The precise effective amount for a subject will depend upon the subject's body weight, size, and health; the nature and extent of the condition; and the therapeutic or combination of therapeutics selected for administration. Therapeutically effective amounts for a given situation can be determined by routine experimentation that is within the skill and judgment of the clinician.

### Formulations

Compositions provided herein comprise pharmaceutically acceptable excipients such as carriers, solvents, stabilizers, adjuvants, diluents, etc., depending upon the particular mode of administration and dosage form. The compositions are generally formulated to achieve a physiologically compatible pH, and range from a pH of about 3 to a pH of about 11, about pH 3 to about pH 7, depending on the formulation and route of administration. The pH can be adjusted to a range from about pH 5.0 to about pH 8. The compositions can comprise a therapeutically effective amount of at least one peptide or peptide conjugate as described herein, together with one or more pharmaceutically acceptable excipients. Optionally, the compositions comprise a combination of the compounds described herein, or can include a second active ingredient useful in the treatment or prevention of bacterial growth (for example and without limitation, anti-bacterial or anti-microbial agents), or can include a combination of peptide or peptide conjugates provided herein.

Suitable excipients include, for example, carrier molecules that include large, slowly metabolized macromolecules such as proteins, polysaccharides, polylactic acids, polyglycolic acids, polymeric amino acids, amino acid copolymers, and inactive virus particles. Other exemplary excipients include antioxidants (for example and without limitation, ascorbic acid), chelating agents (for example and without limitation, EDTA), carbohydrates (for example and without limitation, dextrin, hydroxyalkylcellulose, and hydroxyalkylmethylcellulose), stearic acid, liquids (for example and without limitation, oils, water, saline, glycerol and ethanol) wetting or emulsifying agents, pH buffering substances, and the like.

### Other Terminology and Disclosure

As used herein and in the appended claims, the singular forms “a,” “and,” and “the” include plural referents unless the context clearly dictates otherwise. It is further noted that the claims may be drafted to exclude any element, e.g., any optional element. As such, this statement is intended to serve as antecedent basis for use of such exclusive terminology as “solely,” “only” and the like in connection with the recitation of claim elements, or use of a “negative” limitation.

When a range of values is provided herein, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges, and are also encompassed within the disclosure, subject to any specifically excluded limit in the stated range. Where the stated range includes one or both of the limits, ranges excluding either or both of those included limits are also included in the disclosure.

Unless defined otherwise, all technical and scientific terms used herein have the same meaning as commonly understood by one of ordinary skill in the art to which this disclosure belongs. Any methods and materials similar or equivalent to those described herein can also be used in the practice or testing of the present disclosure.

All publications mentioned herein are incorporated herein by reference to disclose and describe the methods and/or materials for the purpose for which the publications are cited.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order which is logically possible. This disclosure is intended to provide support for all such combinations.

As used herein, “may,” “may comprise,” “may be,” “can,” “can comprise” and “can be” all indicate something envisaged by the inventors that is functional and available as part of the subject matter provided.

### EXAMPLES

While the following examples describe specific embodiments, variations and modifications will occur to those skilled in the art. Accordingly, only such limitations as appear in the claims should be placed on the invention.

### Example 1

### Peptides

Phage display was used to identify candidate peptides specific for EpCAM. A library of M13 bacteriophage (New England Biolabs) was incubated with the extracellular domain of EpCAM using immobilized recombinant proteins to identify high affinity binders.³⁴ Biopanning was performed using a decreasing quantity (100, 80, 60, and 40 μg) of purified protein in each round for increased specificity. The bound phages were eluted, amplified, precipitated, and tittered. Enriched clones were sequenced to identify the most promising candidates. Candidate peptides were synthesized with >95% purity by HPLC, and the experimental mass-to-charge (m/z) ratio was confirmed by mass spectrometry.

The peptides HPDMFTRTHSHN (SEQ ID NO: 1), HGLHSMHNKLQD (SEQ ID NO: 2), GKPAVHYIHLRH (SEQ ID NO: 3), and HPFLHWNYGQRT (SEQ ID NO: 4) were chosen for further development.

### Example 2

### Optimization and Labeling of Peptides

The peptide HPDMFTRTHSHN (SEQ ID NO: 1) was mutated and evaluated using a structural model (4MZV) to optimize binding affinity and specificity to EpCAM.³⁷ Alignment was assessed by rotating the target and ligand about the center of mass over the full range of intermolecular distances and rotational angles.³⁸ Multiple sequences were evaluated to achieve minimum docking energy. A potential energy of Eₜ<−300 was obtained. The optimized sequence was scrambled and evaluated using the structural model for use as control.

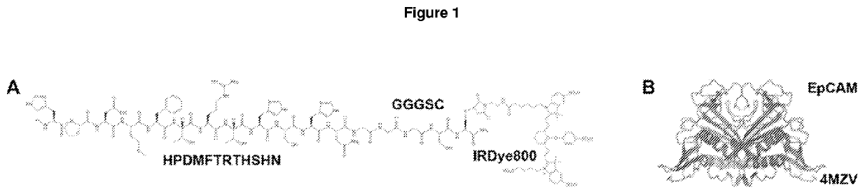

The optimized peptide (referred to herein as HPD*) was then labeled with IRDye800 via a GGGSC linker (SEQ ID NO: 5) to prevent steric hindrance. The labeled, optimized peptide is referred to herein as HPD*-IRDye800. See, FIG. 1A. IRDye800 was chosen for its high quantum yield, photostability, and deep tissue penetration (>1 cm).³⁴ This NIR fluorophore has spectral properties similar to that of ICG, and has been found safe for clinical use.^{35,36}

### Example 3

### Validation of Binding

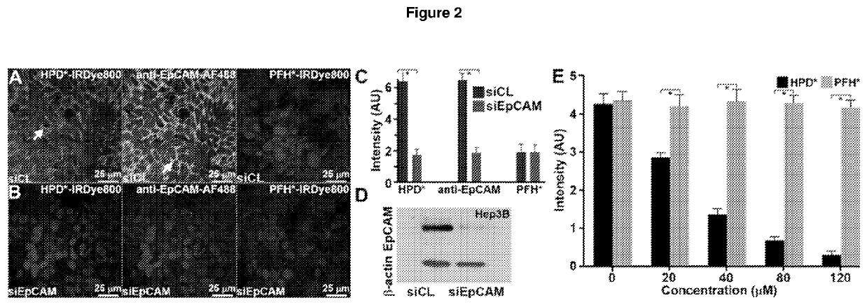

Specific binding of HPD*-IRDye800 to EpCAM was validated using siRNA knockdown in human SG231 (EpCAM+) ICC cells. Preliminary data were obtained using Hep3B (EpCAM+) human HCC cells. The cells were treated with siRNA against EpCAM (siEpCAM) and control siRNA (siCL). Decreased cell surface staining was observed with HPD*-IRDye800 and anti-EpCAM-AF488 for siEpCAM versus siCL but not with scrambled peptide PFH*-IRDye800 (control), FIG. 2A,B. Quantified results show these differences to be significant, FIG. 2C. Western blot shows EpCAM expression for each cell, FIG. 2D. These results predict a power>99% to obtain *P<0.01 for interactions with a 3-fold difference using 10 cells chosen at random from 3 slides.

Specific binding of HPD*-IRDye800 to EpCAM was also validated using a competition assay. Unlabeled HPD* was added to compete for binding with IRDye800-labeled HPD* with concentrations ranging from 0-120 mM. Decreased intensity was observed with increasing concentrations of unlabeled HPD* but not with addition of scrambled peptide PFH* (control), FIG. 2E.

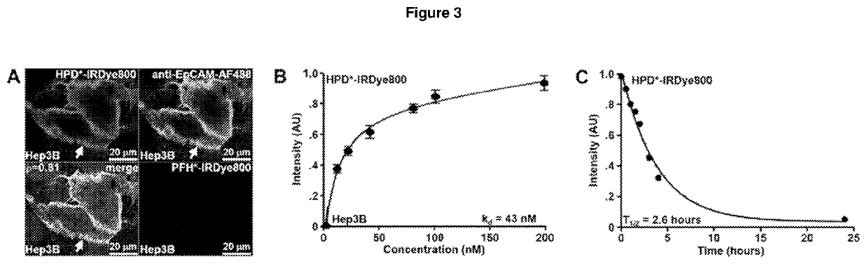

Finally, specific binding of HPD*-IRDye800 to EpCAM was validated using a co-localization assay. Binding by HPD*-IRDye800 and anti-EpCAM-AF488 to the surface (arrows) of Hep3B cells co-localized with a correlation of p=0.81 measured on the merged image, FIG. 3A.

### Example 4

### Binding Affinity

The apparent dissociation constant (k_{d}) of HPD*-IRDye800 was determined to provide a measure of binding affinity.³⁹ HPD*-IRDye800 was incubated with Hep3B cells over concentrations ranging from 0-200 nM, and the fluorescence intensity was measured with flow cytometry. A k_{d}=43 nM was found, FIG. 3B.

### Example 5

### Serum Stability

The serum half-life of HPD*-IRDye800 was measured to assess peptide stability. HPD*-IRDye800 was injected intravenously in live mice. Fluorescence intensities were measured time points ranging from 0-24 hours, and fit to a first order kinetic model. A serum half-life of T_{1/2}=2.6 hours was determined, FIG. 3C.

### Example 6

### EpCAM as a CSC Marker

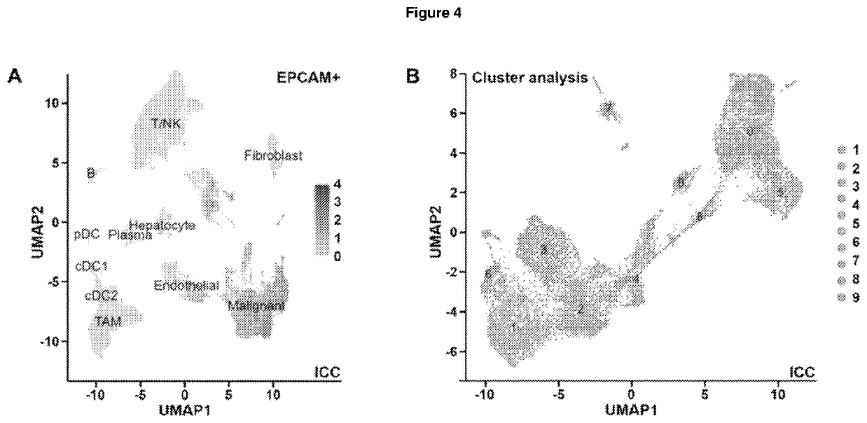

EpCAM as a CSC marker in metastatic lymph nodes was validated by analyzing gene expression from individual cholangiocytes. Data from small duct cholangiocytes harvested from n=15 specimens of primary de novo ICC is shown in FIG. 4A,B. EPCAM was found to be highly expressed in clusters 1 and 6 in addition to cancer stem cell markers THY1 and PROM1.

### Example 7

### Patient-Derived Xenograft (PDX) Tumor Model

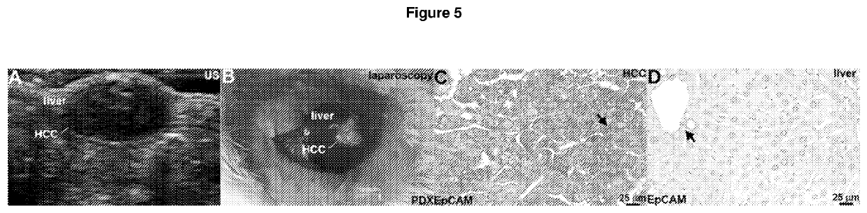

Fresh de novo ICC specimens were obtained from the Michigan Tissue Procurement Core (TPC) and the Peking University People's Hospital bio-bank, respectively. PDX tumors were implanted orthotopically in the liver of NOD Cg-Prkdcll2rgSzJ (NSG) mice. Mutations in scid and a complete null allele of IL2rg^{null} result in extreme immunodeficiency that allows for growth of human ICC tumor specimens.^{40,41} Tumor viability was monitored weekly by ultrasound, FIG. 5A. A representative laparoscopic image showed successful tumor implantation in mouse liver, FIG. 5B. Strong staining for EpCAM was shown from resected human HCC specimens using IHC, FIG. 5C. Normal mouse liver showed minimal staining for EpCAM, FIG. 5D.

### Example 8

### Tumor Uptake of EpCAM-Specific Peptide

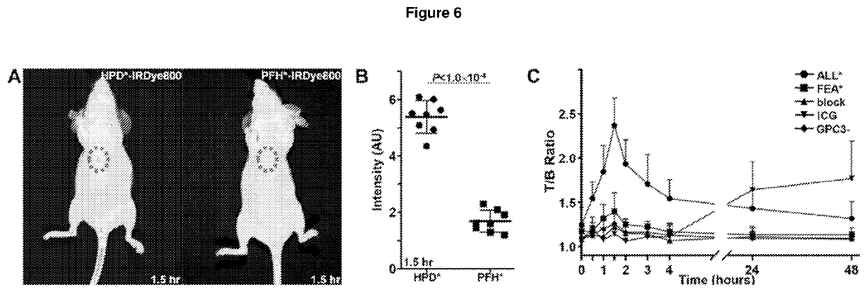

Peak uptake of HPD*-IRDye800 by ICC tumor was measured in vivo using near-infrared (NIR) fluorescence imaging. Data was obtained for HPD*-IRDye800 in human HCC Hep3B xenograft tumors. HPD*-IRDye800 (target) and PFH*-IRDye800 (control) were administered via tail vein in tumor bearing mice. Tumor uptake (dashed red ovals) at 1.5 hours post-injection is shown, FIG. 6A. The mean intensity for HPD*-IRDye800 was significantly greater than that for PFH*-IRDye800 by >3.2-fold, FIG. 6B.

### Example 9

### Pharmacokinetics of EpCAM-Specific Peptide

The time course for peak uptake of HPD*-IRDye800 by ICC tumor was charactized in vivo using NIR fluorescence imaging. Data for HPD*-IRDye800 in human HCC Hep3B xenograft tumors is shown in FIG. 6C. HPD*-IRDye800 (target), PFH*-IRDye800 (control), HPD* (block), and ICG were administered intravenously, and NIR fluorescence images were collected over 48 hours. Blocking was performed by injecting unlabeled HPD* prior to HPD*-IRDye800 to compete for binding and serve as an additional control. Minimal intensity from tumors was seen prior to peptide injection (0 hour). Quantified intensities showed a peak tumor-to-background (T/B) ratio at 1.5 hours after peptide injection. By comparison, ICG did not reach peak uptake until over 48 hours post-injection. This much longer time frame can be explained by non-specific, passive accumulation of ICG in tumor via the enhanced permeability and retention (EPR) effect.⁴² The signal decreased to the baseline by ~48 hours post-injection. For the differences and variances shown, we expect to achieve a power>95% to obtain P<0.01 for target versus all controls by imaging n=8 mice per group.

### Example 10

### Specific Tumor Uptake of EpCAM-Specific Peptide

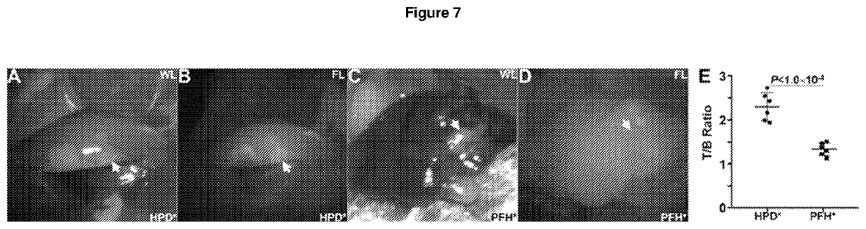

A standard surgical laparoscope was used to collect NIR fluorescence images in vivo to validate specific uptake of HPD*-IRDye800 in the pre-clinical orthotopic PDX model of ICC. Data was obtained for uptake of HPD*-IRDye800 in human HCC cell-derived xenograft tumors. Ultrasound was performed to identify the presence of a viable orthotopic tumor in mouse liver, and to monitor growth. HPD*-IRDye800 and PFH*-IRDye800 were administered systemically ~1.5 hours (peak uptake) prior to imaging. Representative white light (WL) and fluorescence (FL) images were collected in vivo from the exposed liver, FIG. 7A-D. Image intensities were quantified, and the mean T/B ratio was found to be significantly greater for HPD*-IRDye800 versus PFH*-IRDye800 (control) by >1.7-fold, FIG. 7E. For the differences and variances shown, we expect to achieve a power>95% to obtain P<0.01 for the target versus control peptides by imaging n=8 mice per group.

### Example 11

### Specific Peptide Binding Ex Vivo

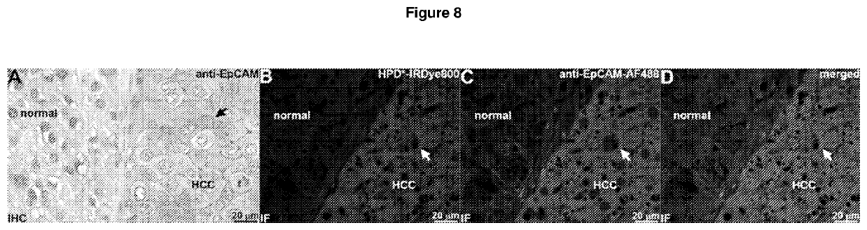

After imaging was completed as described in Example 6, the mice were euthanized, and the livers were resected, formalin-fixed, and sectioned to confirm EpCAM expression in human ICC tumors. IHC was performed using anti-EpCAM antibody, and strong reactivity was observed to HCC (arrow) but not to normal mouse liver, FIG. 8A. Immunofluorescence was performed, and strong staining with HPD*-IRDye800 and anti-EpCAM-AF488 was seen to the surface of HCC tumor cells (arrows) but not to normal mouse liver, FIG. 8B,C. The merged image confirms co-localization of peptide and antibody binding to HCC tumor but not to normal mouse liver, FIG. 8D.

### Example 12

### Peptide Biodistribution

Uptake of HPD*-IRDye800 and control peptides in PDX HCC tumor and non-tumor tissues was assessed following intravenous administration. ICG was used as a control. The mice were euthanized at the time frame for peak uptake post-injection. The tumor and major organs, including spleen, kidney, stomach, liver, intestine, heart, lung, and brain, were excised and imaged using the Pearl Trilogy (LI-COR Biosciences) with excitation at λₑₓ=785 nm and emission at λₑₘ=820 nm. Fluorescence intensities were quantified from the tumor and other organs using Image Studio software.

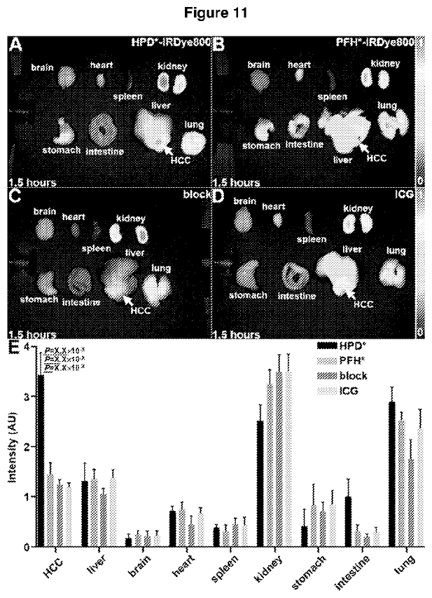

Quantified results showed uptake of HPD*-IRDye800 by the PDX HCC tumor was significantly higher than that for the other organs (FIG. 11).

### Example 13

### Peptide Toxicity

Tumor bearing mice were euthanized on day 0 and 15 post-injection of the HPD*-IRDye800 and control peptides. Whole blood (~600 μL) was collected by cardiac puncture and submitted for standard hematology and chemistry. Major organs, including liver, kidney, heart, lung, spleen, stomach, intestine, and brain were resected, processed, and evaluated as routine histology (H&E). A total of 8 mice in each cohort were evaluated.

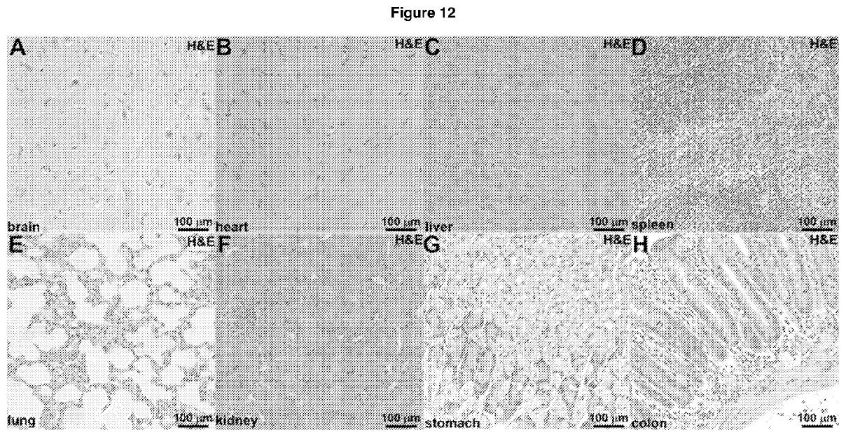

No signs of acute toxicity were seen on histology (H&E) of vital organs, including brain, heart, liver, spleen, lung, kidney, stomach and colon (FIG. 12).

### Example 14

### EpCAM as Imaging Target for HCC

Immunohistochemistry (IHC) was performed to detect EpCAM expression in a total of n=57 human ICC specimens. Formalin-fixed paraffin-embedded (FFPE) specimens were processed using standard methods, including deparaffinization and antigen unmasking.

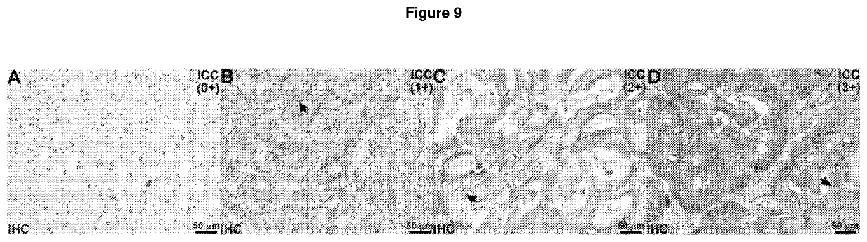

The ICC specimens were evaluated for anti-EpCAM immunoreactivity using a standard IHC scoring system, and found none (0+), minimal (1+), moderate (2+), and strong (3+) reactivity in a total of 4 (7%), 8 (14%), 24 (42%), and 21 (37%) specimens, respectively, resulting in a total of 53 (93%) positives, FIG. 9A-D.

### Example 15

### Specific Peptide Binding to Human HCC

Binding of HPD*-IRDye800 to human HCC specimens and metastatic lymph nodes ex vivo was examined using immunofluorescence.

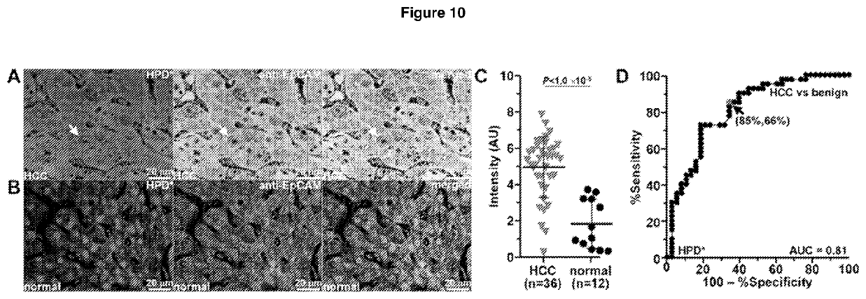

Intense staining of HCC with HPD*-IRDye800 (red) and anti-EpCAM-AF488 (green) was seen at the cell surface (arrows), FIG. 10A. Substantially reduced intensity was seen to sections of normal human liver for both the peptide and antibody, FIG. 10B. The merged images show co-localization of peptide and antibody binding to HCC and normal liver, and a Pearson's correlation coefficient of p=0.68 and 0.66, respectively, were calculated. The fluorescence intensities were quantified, and the mean (±SD) value for HPD*-IRDye800 was found to be significantly greater for binding to HCC than to normal human liver, FIG. 10C. An ROC curve shows 85% sensitivity and 66% specificity for distinguishing HCC from normal human liver with an AUC=0.81, FIG. 10D. Results were compared with histology evaluated by an expert liver pathologist.

### Example 16

### In Vivo Whole-Body Fluorescence Imaging

Tumor-bearing mice [a patient-derived xenograft (PDX) tumor model of HCC] were injected intravenously with the HPD*-IRDye800 and control peptides (300 μM in 200 μl PBS). Unlabeled peptide and ICG were administrated as above. The spatial extent and margins of tumors were identified using a NIR whole-body fluorescence imaging system (Pearl®, LI-COR Biosciences) up to 24 h post injection. The images were acquired using λₑₓ=800 nm with 85 μm resolution and 16.8×12 cm² field of view (FOV). Image Studio software (Li-Cor Biosciences) was used for analysis. Regions of interest (ROI) with area equal to that of the tumor and adjacent in location was measured for background.

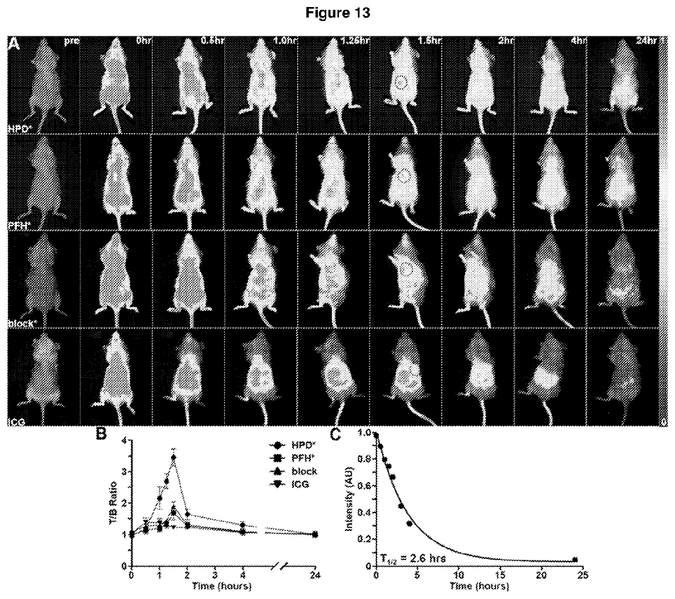

Specific binding by HPD*-IRDye800 was seen in the in vivo images showing greater uptake in comparison with the control peptide PFH*, block, and non-specific ICG (FIG. 13).

### Example 17

### Detection of Liver Micrometastases

Laparoscopic images of the liver were collected in vivo from the PDX tumor model mice using a custom imaging module attached to standard surgical laparoscope. Immunohistochemistry using anti-cytokeratin confirmed presence of human tumor. White light (WL) and fluorescence (FL) images of liver were collected at 1.5 hours post-injection of HPD*-IRDye800.

Immunofluorescence images also were collected from tissue sections of the PDX liver tumors.

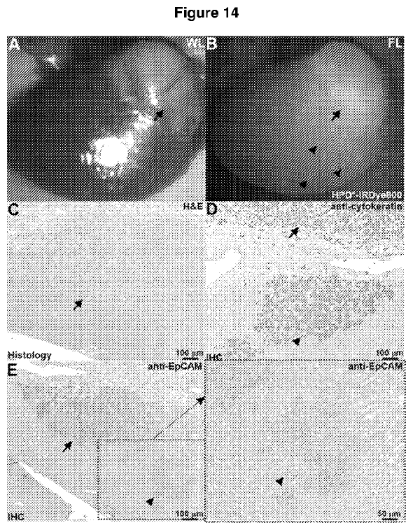

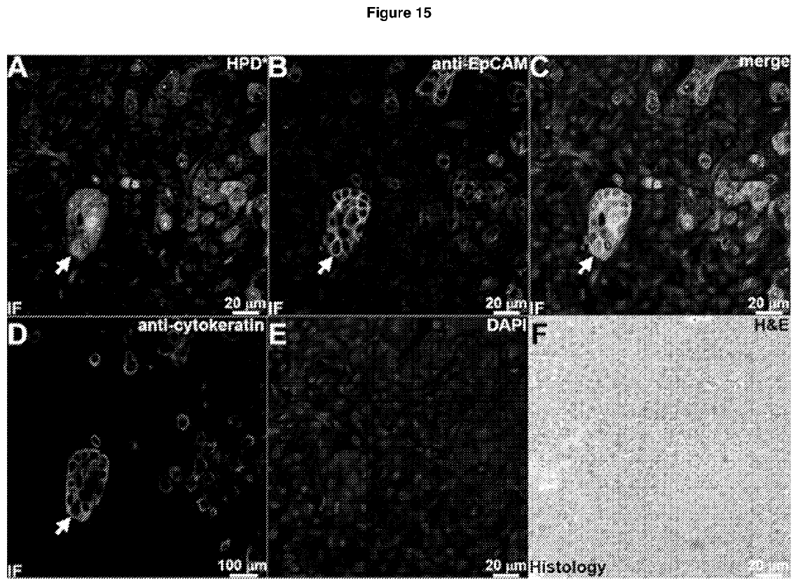

HPD*-IRDye800 detected very small tumor volumes in the form of micrometastases within the liver (FIGS. 14 and 15).

### Example 18

### Detection of Lung Micrometastases

Immunofluorescence images were also collected from lung tissue sections of the PDX tumor model of HCC mice.

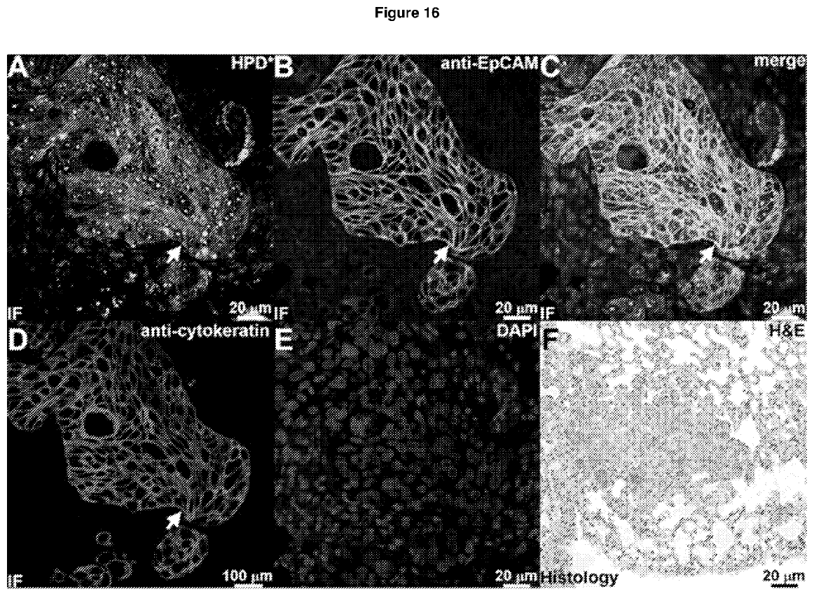

HPD*-IRDye800 detected very small tumor volumes in the form of micrometastases to the mouse lung (FIG. 16).

### Document List

All documents cited in this application are hereby incorporated by reference in their entirety, with particular attention to the disclosure for which they are referred.
1. Florio A A, Ferlay J, Znaor A, Ruggieri D, Alvarez C S, Laversanne M, Bray F, McGlynn K A, Petrick J L. Global trends in intrahepatic and extrahepatic cholangiocarcinoma incidence from 1993 to 2012. Cancer. 2020; 126:2666-2678. PMC7323858.
2. Sirica A E, Gores G J, Groopman J D, Selaru F M, Strazzabosco M, Wei Wang X, Zhu A X. Intrahepatic Cholangiocarcinoma: Continuing Challenges and Translational Advances. Hepatology. 2019; 69:1803-1815. PMC6433548.
3. Bertuccio P, Malvezzi M, Carioli G, Hashim D, Boffetta P, El-Serag H B, La Vecchia C, Negri E. Global trends in mortality from intrahepatic and extrahepatic cholangiocarcinoma. J Hepatol 2019; 71:104-114. PMID: 30910538.
4. Rizvi S, Khan S A, Hallemeier C L, Kelley R K, Gores G J. Cholangiocarcinoma-evolving concepts and therapeutic strategies. Nat Rev Clin Oncol 2018; 15:95-111. PMC5819599.
5. Hewitt D B, Brown Z J, Pawlik T M. Surgical management of intrahepatic cholangiocarcinoma. Expert Rev Anticancer Ther 2022; 22:27-38. PMID: 34730474.
6. Wu L, Tsilimigras D I, Paredes A Z, Mehta R, Hyer J M, Merath K, Sahara K, Bagante F, Beal E W, Shen F, Pawlik T M. Trends in the Incidence, Treatment and Outcomes of Patients with Intrahepatic Cholangiocarcinoma in the USA: Facility Type is Associated with Margin Status, Use of Lymphadenectomy and Overall Survival. World J Surg 2019; 43:1777-1787. PMID: 30820734.
7. Guro H, Kim J W, Choi Y, Cho J Y, Yoon Y S, Han H S. Multidisciplinary management of intrahepatic cholangiocarcinoma: Current approaches. Surg Oncol. 2017; 26:146-152. PMID: 28577720.
8. Twohig P, Peeraphatdit T B, Mukherjee S. Current status of liver transplantation for cholangiocarcinoma. World J Gastrointest Surg 2022; 14:1-11. PMC8790328.
9. El-Diwany R, Pawlik T M, Ejaz A. Intrahepatic Cholangiocarcinoma. Surg Oncol Clin N Am 2019; 28:587-599. PMID: 31472907.
10. Lee T K, Guan X Y, Ma S. Cancer stem cells in hepatocellular carcinoma—from origin to clinical implications. Nat Rev Gastroenterol Hepatol 2022; 19:26-44. PMID: 34504325.
11. Kokuryo T, Yokoyama Y, Nagino M. Recent advances in cancer stem cell research for cholangiocarcinoma. J Hepatobiliary Pancreat Sci 2012; 19:606-13. PMID: 22907641.
12. Chen D, Li Z, Cheng Q, Wang Y, Qian L, Gao J, Zhu J Y. Genetic alterations and expression of PTEN and its relationship with cancer stem cell markers to investigate pathogenesis and to evaluate prognosis in hepatocellular carcinoma. J Clin Pathol 2019; 72:588-596. PMID: 31126975.
13. Ma Y C, Yang J Y, Yan L N. Relevant markers of cancer stem cells indicate a poor prognosis in hepatocellular carcinoma patients: a meta-analysis. Eur J Gastroenterol Hepatol 2013; 25:1007-16. PMID: 23478672.
14. Safarikia S, Carpino G, Overi D, Cardinale V, Venere R, Franchitto A, Onori P, Alvaro D, Gaudio E. Distinct EpCAM-Positive Stem Cell Niches Are Engaged in Chronic and Neoplastic Liver Diseases. Front Med 2020; 7:479. PMC7492539.
15. Carpino G, Cardinale V, Renzi A, Hov J R, Berloco P B, Rossi M, Karlsen T H, Alvaro D, Gaudio E. Activation of biliary tree stem cells within peribiliary glands in primary sclerosing cholangitis. J Hepatol 2015; 63:1220-8. PMID: 26119688.
16. de Jong I E M, Matton A P M, van Praagh J B, van Haaften W T, Wiersema-Buist J, van Wijk L A, Oosterhuis D, Iswandana R, Suriguga S, Overi D, Lisman T, Carpino G, Gouw A S H, Olinga P, Gaudio E, Porte R J. Peribiliary Glands Are Key in Regeneration of the Human Biliary Epithelium After Severe Bile Duct Injury. Hepatology 2019; 69:1719-1734. PMC6594148.
17. Baeuerle P A, Gires O. EpCAM (CD326) finding its role in cancer. Br J Cancer 2007; 96:417-23. PMC2360029.
18. Yamashita T, Budhu A, Forgues M, Wang X W. Activation of hepatic stem cell marker EpCAM by Wnt-beta-catenin signaling in hepatocellular carcinoma. Cancer Res 2007; 67:10831-9. PMID: 18006828.
19. Munz M, Baeuerle P A, Gires O. The emerging role of EpCAM in cancer and stem cell signaling. Cancer Res 2009; 69:5627-9. PMID: 19584271.
20. Sulpice L, Rayar M, Turlin B, Boucher E, Bellaud P, Desille M, Meunier B, Clément B, Boudjema K, Coulouarn C. Epithelial cell adhesion molecule is a prognosis marker for intrahepatic cholangiocarcinoma. J Surg Res 2014; 192:117-23. PMID: 24909871.
21. Padthaisong S, Thanee M, Namwat N, Phetcharaburanin J, Klanrit P, Khuntikeo N, Titapun A, Sungkhamanon S, Saya H, Loilome W. Overexpression of a panel of cancer stem cell markers enhances the predictive capability of the progression and recurrence in the early stage cholangiocarcinoma. J Transl Med 2020; 18:64. PMC7008521.
22. Kim G J, Kim H, Park Y N. Increased expression of Yes-associated protein 1 in hepatocellular carcinoma with stemness and combined hepatocellular-cholangiocarcinoma. PLOS One 2013; 8: e75449. PMC3782432.
23. Safarikia S, Carpino G, Overi D, Cardinale V, Venere R, Franchitto A, Onori P, Alvaro D, Gaudio E. Distinct EpCAM-Positive Stem Cell Niches Are Engaged in Chronic and Neoplastic Liver Diseases. Front Med 2020; 7:479. PMC7492539.
24. Wang A, Wu L, Lin J, Han L, Bian J, Wu Y, Robson S C, Xue L, Ge Y, Sang X, Wang W, Zhao H. Whole-exome sequencing reveals the origin and evolution of hepato-cholangiocarcinoma. Nat Commun 2018; 9:894. PMC5832792.
25. Wakizaka K, Yokoo H, Kamiyama T, Kakisaka T, Ohira M, Tani M, Kato K, Fujii Y, Sugiyama K, Nagatsu A, Shimada S, Orimo T, Kamachi H, Matsuoka R, Taketomi A. CD133 and epithelial cell adhesion molecule expressions in the cholangiocarcinoma component are prognostic factors for combined hepatocellular cholangiocarcinoma. Hepatol Res 2020; 50:258-267. PMID: 31661725.
26. Xu J, Sasaki M, Harada K, Sato Y, Ikeda H, Kim J H, Yu E, Nakanuma Y. Intrahepatic cholangiocarcinoma arising in chronic advanced liver disease and the cholangiocarcinomatous component of hepatocellular cholangiocarcinoma share common phenotypes and cholangiocarcinogenesis. Histopathology 2011; 59:1090-9. PMID: 22175889.
27. Zhang Z, He K, Chi C, Hu Z, Tian J. Intraoperative fluorescence molecular imaging accelerates the coming of precision surgery in China. Eur J Nucl Med Mol Imaging 2022. PMID: 35230491.
28. Wang Q, Li X, Qian B, Hu K, Liu B. Fluorescence imaging in the surgical management of liver cancers: Current status and future perspectives. Asian J Surg 2021: S1015-9584 (21) 00567-4. PMID: 34656410.
29. Liu Y, Gao B, Fang C, Su S, Yang X, Tian J, Li B. Application of Near-Infrared Fluorescence Imaging Technology in Liver Cancer Surgery. Surg Innov 2021:1553350621997777. PMID: 33634713.
30. Alam I S, Steinberg I, Vermesh O, van den Berg N S, Rosenthal E L, van Dam G M, Ntziachristos V, Gambhir S S, Hernot S, Rogalla S. Emerging Intraoperative Imaging Modalities to Improve Surgical Precision. Mol Imaging Biol 2018; 20:705-715. PMID: 29916118.
31. Choti M A, Kaloma F, de Oliveira M L, Nour S, Garrett-Mayer E S, Sheth S, Pawlik T M. Patient variability in intraoperative ultrasonographic characteristics of colorectal liver metastases. Arch Surg 2008; 143:29-34. PMID: 18209150.
32. Panarelli, Nicole C. “Intraoperative Evaluation of the Liver, Extrahepatic Bile Ducts, Gallbladder, and Pancreas.” Frozen Section Pathology. Springer, Cham, 2021. 49-100.
33. Smith G P, Scott J K. Libraries of peptides and proteins displayed on filamentous phage. Methods Enzymol 1993; 217:228-257. PMID: 7682645.
34. Ash C, Dubec M, Donne K, Bashford T. Effect of wavelength and beam width on penetration in light-tissue interaction using computational methods. Lasers Med Sci. 2017; 32:1909-1918. PMC5653719.
35. Rosenthal E L, Warram J M, de Boer E, Basilion J P, Biel M A, Bogyo M, Bouvet M, Brigman B E, Colson Y L, DeMeester S R, Gurtner G C, Ishizawa T, Jacobs P M, Keereweer S, Liao J C, Nguyen Q T, Olson J M, Paulsen K D, Rieves D, Sumer B D, Tweedle M F, Vahrmeijer A L, Weichert J P, Wilson B C, Zenn M R, Zinn K R, van Dam G M. Successful Translation of Fluorescence Navigation During Oncologic Surgery: A Consensus Report. J Nucl Med 2016; 57:144-50. PMC4772735.
36. Marshall M V, Draney D, Sevick-Muraca E M, Olive D M. Single-dose intravenous toxicity study of IRDye 800C W in Sprague-Dawley rats. Mol Imaging Biol 2010; 12:583-94. PMC2978892.
37. Pavšič M, Gunčar G, Djinović-Carugo K, Lenarčič B. Crystal structure and its bearing towards an understanding of key biological functions of EpCAM. Nat Commun 2014; 5:4764. PMID: 25163760.
38. Macindoe G, Mavridis L, Venkatraman V, Devignes M D, Ritchie D W. HexServer: an FFT-based protein docking server powered by graphics processors. Nucleic Acids Research 2010; 38 (S2): W445-W449.
39. Thomas R, Chen J, Roudier M M, Vessella R L, Lantry L E, Nunn A D. In vitro binding evaluation of ¹⁷⁷Lu-AMBA, a novel ¹⁷⁷Lu-labeled GRP-R agonist for systemic radiotherapy in human tissues. Clin Exp Metastasis. 2009; 26:105-19. PMID: 18975117.
40. Coughlan A M, Harmon C, Whelan S, O'Brien E C, O'Reilly V P, Crotty P, Kelly P, Ryan M, Hickey F B, O'Farrelly C, Little M A. Myeloid Engraftment in Humanized Mice: Impact of Granulocyte-Colony Stimulating Factor Treatment and Transgenic Mouse Strain. Stem Cells Dev 2016; 25:530-41. PMID: 26879149.
41. Shultz L D, Lyons B L, Burzenski L M, Gott B, Chen X, Chaleff S, Kotb M, Gillies S D, King M, Mangada J, Greiner D L, Handgretinger R. Human lymphoid and myeloid cell development in NOD/LtSz-scid IL2R gamma null mice engrafted with mobilized human hemopoietic stem cells. J Immunol 2005; 174:6477-89. PMID: 15879151.
42. Maeda H, Wu J, Sawa T, Matsumura Y, Hori K. Tumor vascular permeability and the EPR effect in macromolecular therapeutics: a review. J Control Release 2000; 65:271-84. PMID: 10699287.

We claim:

1. A peptide conjugate comprising the peptide HPDMFTRTHSHN (SEQ ID NO: 1), the peptide HGLHSMHNKLOD (SEQ ID NO: 2), the peptide GKPAVHYIHLRH (SEQ ID NO: 3), or the peptide HPFLHWNYGQRT (SEQ ID NO: 4), or a multimer form of the peptide,
wherein the peptide specifically binds to EpCAM and
wherein at least one detectable label, at least one therapeutic moiety, or both, are attached to the peptide or a multimer form of the peptide.

2. The conjugate of claim 1 comprising at least one detectable label attached to the peptide.

3. The conjugate of claim 2 wherein the detectable label is detectable by optical, photoacoustic, ultrasound, positron emission tomography or magnetic resonance imaging.

4. The conjugate of claim 3 wherein the label detectable by optical imaging is fluorescein isothiocyanate (FITC), Cy5, Cy5.5 or IRdye800.

5. The conjugate of claim 4 wherein the label is IRdye 800.

6. The conjugate of claim 3 wherein the label detectable by magnetic resonance imaging is gadolinium, Gd-DOTA or an iron oxide nanoparticle.

7. The conjugate of claim 6 wherein the label is Gd-DOTA.

8. The conjugate of claim 1 wherein the multimer form of the peptide is a dimer formed with an aminohexanoic acid linker.

9. The conjugate of claim 2 wherein the detectable label is attached to the peptide by a peptide linker.

10. The conjugate of claim 9 wherein a terminal amino acid of the linker is lysine.

11. The conjugate of claim 10 wherein the linker comprises the sequence GGGSK set out in SEQ ID NO: 5.

12. The conjugate of 1, 2, 3, 4, 5, 6, 7, 8, 9, 10 or 11 comprising at least one therapeutic moiety attached to the peptide.

13. The conjugate of claim 12 wherein the therapeutic moiety is chemotherapeutic agent.

14. The conjugate of claim 12 wherein the therapeutic moiety is a polymeric nanoparticle or micelle.

15. The conjugate of claim 13 wherein the micelle is an octadecyl lithocholate micelle.

16. The conjugate of claim 15 wherein the nanoparticle or micelle is pegylated.

17. The conjugate of claim 13 wherein the nanoparticle or micelle encapsulates carboplatin, paclitaxel, cisplatin, 5-fluorouracil (5-FU), oxaliplatin, capecitabine, irinotecan chlorambucil or sorafenib.

18. A composition comprising the conjugate of claim 1, 2, 3, 4, 5, 6, 7, 8, 9, 10, 11, 12, 13, 14, 15, 16 or 17 and a pharmaceutically acceptable excipient.

19. A method for detecting epithelial cell-derived cancer cells in a patient comprising the steps of administering the conjugate of claim 1, 2, 3, 4, 5, 6, 7, 8, 9, 10 or 11 to the patient and detecting binding of the conjugate to cells.

20. A method for treating an epithelial cell-derived cancer comprising administering to a patient a peptide reagent of claim 12, 13, 14, 15, 16 or 17.

21. A method of determining the effectiveness of a treatment for an epithelial cell-derived cancer in a patient comprising the step of administering the conjugate of claim 1, 2, 3, 4, 5, 6, 7, 8, 9, 10 or 11 to the patient, visualizing a first amount of cells labeled with the conjugate, and comparing the first amount to a previously-visualized second amount of cells labeled with the conjugate,
wherein a decrease in the first amount cells labeled relative to the previously-visualized second amount of cells labeled is indicative of effective treatment.

22. The method of claim 21 further comprising obtaining a biopsy of the cells labeled by the conjugate.

23. A method for delivering a therapeutic moiety to epithelial cell-derived cancer cells of a patient comprising the step of administering the conjugate of claim 12 to the patient.

24. The method of claim 19 wherein the cancer cells are intrahepatic cholangiocarcinoma (ICC) cells, hepatocellular carcinoma (HCC) cells, breast cancer cells, colon cancer cells or basal cell carcinoma of the skin cells.

25. The method of claim 20 wherein the cancer cells are intrahepatic cholangiocarcinoma (ICC) cells, hepatocellular carcinoma (HCC) cells, breast cancer cells, colon cancer cells or basal cell carcinoma of the skin cells.

26. The method of claim 21 wherein the cancer cells are intrahepatic cholangiocarcinoma (ICC) cells, hepatocellular carcinoma (HCC) cells, breast cancer cells, colon cancer cells or basal cell carcinoma of the skin cells.

27. The method of claim 22 wherein the cancer cells are intrahepatic cholangiocarcinoma (ICC) cells, hepatocellular carcinoma (HCC) cells, breast cancer cells, colon cancer cells or basal cell carcinoma of the skin cells.

28. The method of claim 23 wherein the cancer cells are intrahepatic cholangiocarcinoma (ICC) cells, hepatocellular carcinoma (HCC) cells, breast cancer cells, colon cancer cells or basal cell carcinoma of the skin cells.

29. A kit for administering the composition of claim 18 to a patient in need thereof, said kit comprising the composition of claim 18, instructions for use of the composition and a device for administering the composition to the patient.

30. An EpCAM-specific peptide consisting of the amino acid sequence HPDMFTRTHSHN (SEQ ID NO: 1), HGLHSMHNKLQD (SEQ ID NO: 2), GKPAVHYIHLRH (SEQ ID NO: 3), or HPFLHWNYGQRT (SEQ ID NO: 4).
